(19) United States
(12) Patent Application Publication
(10) Pub. No.: US 2022/0172358 A1
(43) Pub. Date: Jun. 2, 2022

(54) IMAGE PROCESSING APPARATUS, IMAGE PROCESSING METHOD, AND RECORDING MEDIUM (as amended)

(71) Applicant: NIKON CORPORATION, Minato-ku, Tokyo (JP)

(72) Inventor: Mariko MUKAI, Minato-ku, Tokyo (JP)

(73) Assignee: NIKON CORPORATION, Minato-ku, Tokyo (JP)

(21) Appl. No.: 17/600,346

(22) PCT Filed: Apr. 25, 2019
(86) PCT No.: PCT/JP2019/017728

§ 371 Date: Sep. 30, 2021

(51) Int. Cl. G06T 7/00; A61B 3/12

(52) U.S. Cl. CPC G06T 7/0012; A61B 3/1233; G06T 2207/30104; G06T 2207/10101; G06T 2207/30041

(57) ABSTRACT

An image processing apparatus includes: an acquisition unit that acquires first angiographic image data of a subject eye, and second angiographic image data of the subject eye generated after the first angiographic image data; a first generation unit that calculates a first blood vessel area density from the first angiographic image data to generate first blood vessel area density map data based on the first blood vessel area density, and that calculates a second blood vessel area density from the second angiographic image data to generate second blood vessel area density map data based on the second blood vessel area density; a second generation unit that generates comparison image data for comparing the first blood vessel area density map data to the second blood vessel area density map data; and an output unit that outputs the comparison image data.

### BACKGROUND

The present invention relates to an image processing apparatus, an image processing method, and an image processing program.

JP 2017-77414 A discloses an ophthalmic analysis device for analyzing subject eye data including blood vessel information of a subject eye. However, J P 2017-77414 A does not take into consideration ease of follow-up observation of a lesion using an image generated through optical coherence tomography angiography (hereinafter referred to as “OCT-A”).

### SUMMARY

First aspect of the disclosure in this application is an image processing apparatus, comprising: an acquisition unit that acquires first angiographic image data of a subject eye, and second angiographic image data of the subject eye generated after the first angiographic image data; a first generation unit that calculates a first blood vessel area density from the first angiographic image data to generate first blood vessel area density map data based on the first blood vessel area density, and that calculates a second blood vessel area density from the second angiographic image data to generate second blood vessel area density map data based on the second blood vessel area density; a second generation unit that generates comparison image data for comparing the first blood vessel area density map data to the second blood vessel area density map data; and an output unit that outputs the comparison image data.

Second aspect of the disclosure in this application is an image processing method, wherein a processor executes: acquisition processing for acquiring first angiographic image data of a subject eye, and second angiographic image data of the subject eye generated after the first angiographic image data; first generation processing for calculating a first blood vessel area density from the first angiographic image data to generate first blood vessel area density map data based on the first blood vessel area density, and for calculating a second blood vessel area density from the second angiographic image data to generate second blood vessel area density map data based on the second blood vessel area density; second generation processing for generating comparison image data for comparing the first blood vessel area density map data to the second blood vessel area density map data; and output processing for outputting the comparison image data.

### BRIEF DESCRIPTION OF THE DRAWINGS

FIG. 1 is a descriptive drawing showing a generation example 1 of heat map data of the eye fundus before and after treatment by photodynamic therapy (PDT) performed on a subject eye of a patient suffering from age-related macular degeneration.

FIG. 2 is a descriptive drawing showing a generation example 2 of heat map data 103 of the eye fundus before and after treatment through PDT performed on a subject eye of a patient suffering from age-related macular degeneration.

FIG. 3 is a descriptive view showing a configuration example of an ophthalmic system.

FIG. 4 is a block diagram for illustrating a hardware configuration example of a computer.

FIG. 5 is a block diagram showing a functional configuration example of the image processing apparatus.

FIG. 6 is a flowchart showing an example of image processing steps executed by the image processing apparatus.

FIG. 7 is a descriptive drawing showing a display screen example.

### DETAILED DESCRIPTION OF THE EMBODIMENTS

<Generation Example of Pre- and Post-Treatment Heat Map Data>

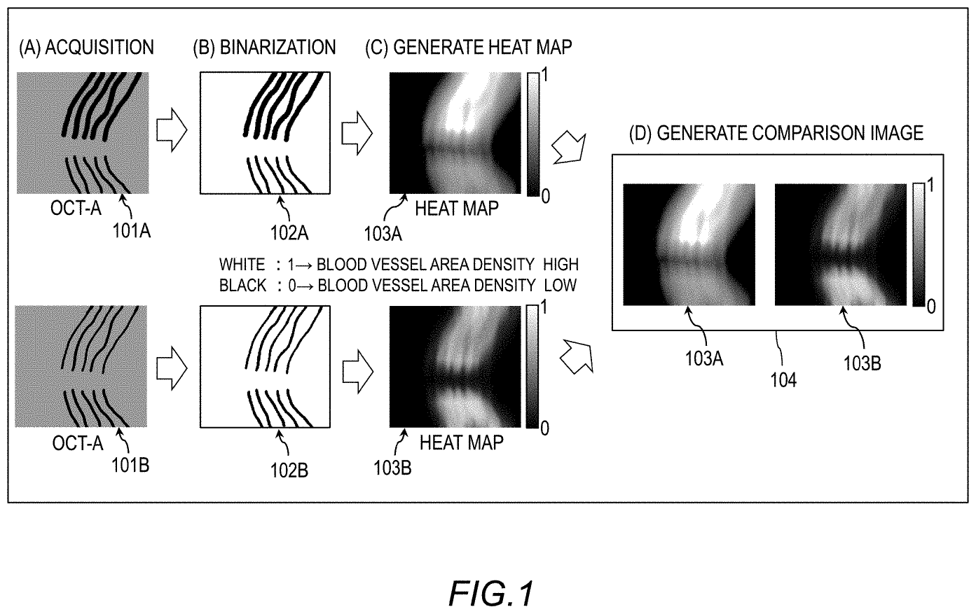

FIG. 1 is a descriptive drawing showing a generation example 1 of heat map data of the eye fundus before and after treatment by photodynamic therapy (PDT) performed on a subject eye of a patient suffering from age-related macular degeneration. Photodynamic therapy (PDT) is a treatment in which a drug (visudyne) that reacts to a laser beam is intravenously injected into the patient's body, after which a lesion is irradiated with a weak laser beam.

The capital letter “A” suffixes on the reference characters of the image data indicates that the imaging time is earlier than image data with suffixes of “B” on the reference characters thereof regardless of whether treatment has been conducted. In this example, image data with a suffix of “A” on the reference character is image data prior to treatment, and image data with a suffix of “B” on the reference character is image data after treatment.

(A) indicates an image processing example in an image processing apparatus. The image processing apparatus acquires first angiographic image data 101A as subject eye image data prior to treatment. Also, the image processing apparatus acquires second angiographic image data 101B as subject eye image data after treatment of the same subject eye of a patient. If not distinguishing between the first angiographic image data 101A and the second angiographic image data 101B, these are simply referred to collectively as angiographic image data 101.

The image processing apparatus scans 3-dimensional OCT image data of the same position of the subject eye a plurality of times to detect a change over time in the blood flow, and generates 3-dimensional angiographic image data (OCT-angiography, OCT-A image data) in which the blood vessels are emphasized. A flat image (en face image) generated by cutting out a two-dimensional plane at the depth of the choroid from the 3-dimensional angiographic image data is the angiographic image data 101. That is, the angiographic image data 101 is choroid blood vessel image data in which the choroid blood vessels are made visible.

(B) The image processing apparatus subjects the first angiographic image data 101A from (A) to binarization processing, and generates first angiographic image data 102A that has been binarized. Also, the image processing apparatus subjects the second angiographic image data 101B from (A) to binarization processing, and generates second angiographic image data 102B that has been binarized. If not distinguishing between the binarized first angiographic image data 102A and the binarized second angiographic image data 102B, these are simply referred to collectively as binarized angiographic image data 102.

(C) The image processing apparatus calculates a first blood vessel area density from the binarized first angiographic image data 102A of (B), and generates first blood vessel area density map data based on the first blood vessel area density. In the present embodiment, first heat map data (hereinafter referred to as “heat map data 103A”) in which the blood vessel area density values are represented in grayscale or color is generated as the first blood vessel area density map data.

Also, the image processing apparatus calculates a second blood vessel area density from the binarized second angiographic image data 102B of (B), and generates second blood vessel area density map data based on the second blood vessel area density. In the present embodiment, second heat map data (hereinafter referred to as “heat map data 103B”) in which the second blood vessel area density values are represented in grayscale or color is generated as the second blood vessel area density map data. If not distinguishing between the heat map data 103A based on the second blood vessel area density and the heat map data 103B based on the second blood vessel area density, these are simply referred to collectively as heat map data 103 based on the blood vessel area density.

The same scale is used for the grayscale or color representation for generating the heat map data 103A and the heat map data 103B. That is, the same blood vessel area density values in the heat map data 103A and the heat map data 103B are represented with the same colors. If not distinguishing between first blood vessel area density and the second blood vessel area density, these are simply referred to collectively as the blood vessel area density. If similarly not distinguishing between the first blood vessel area density map data and the second blood vessel area density map data, these are simply referred to collectively as blood vessel area density map data.

The blood vessel area density is the proportion of pixels depicting blood vessels within a region of a given size (e.g., 100 pixels×100 pixels). The image processing apparatus calculates the overall blood vessel area density of the angiographic image data 102 by performing averaging filter processing (details described later) performed on the binarized angiographic image data 102.

The image processing apparatus generates the heat map data 103 as the blood vessel area density map data on the basis of the blood vessel area density. The heat map data is image data in which regions are filled with colors corresponding to the blood vessel area density values. In FIG. 1, the darker (whiter) the color is, the higher the blood vessel area density indicated is (similarly applies to subsequent drawings). However, the configuration is not limited to a heat map format in which differences in the blood vessel area density are represented by different colors, and image data that represents the blood vessel area density through contour lines or image data that represents the blood vessel area densities as numerical values may be used.

(D) The image processing apparatus combines the heat map data 103A and the heat map data 103B of (C) to generate comparison image data 104. The comparison image data 104 is image data including the heat map data 103A and the heat map data 103B.

As a result, the comparison image data 104 is displayed by the image processing apparatus or the output destination of the comparison image data 104. Thus, when the comparison image data 104 is displayed, users such as physicians can compare the heat map data 103A to the heat map data 103B and observe therapeutic effects.

The aim of photodynamic therapy (PDT) is to restore dilated blood vessels to a normal diameter. Through comparison of the heat map data 103 before and after PDT (observing the difference in colors in the heat map data), it is possible to observe that dilated blood vessels prior to treatment have been restored to a normal diameter after treatment.

The aim of treatment of exudative age-related macular degeneration, central serous chorioretinopathy, or the like using an anti-VEGF agent is to reduce new blood vessels. It is also possible for the user to observe that while there were new blood vessels before treatment, the new blood vessels were reduced after treatment in the area where a therapeutic effect was attained by seeing the difference in colors in the heat map data 103. Another effect exhibited by this configuration is that it is possible to observe not only therapeutic effects but also worsening of symptoms (dilation of blood vessels, formation of new blood vessels, etc.).

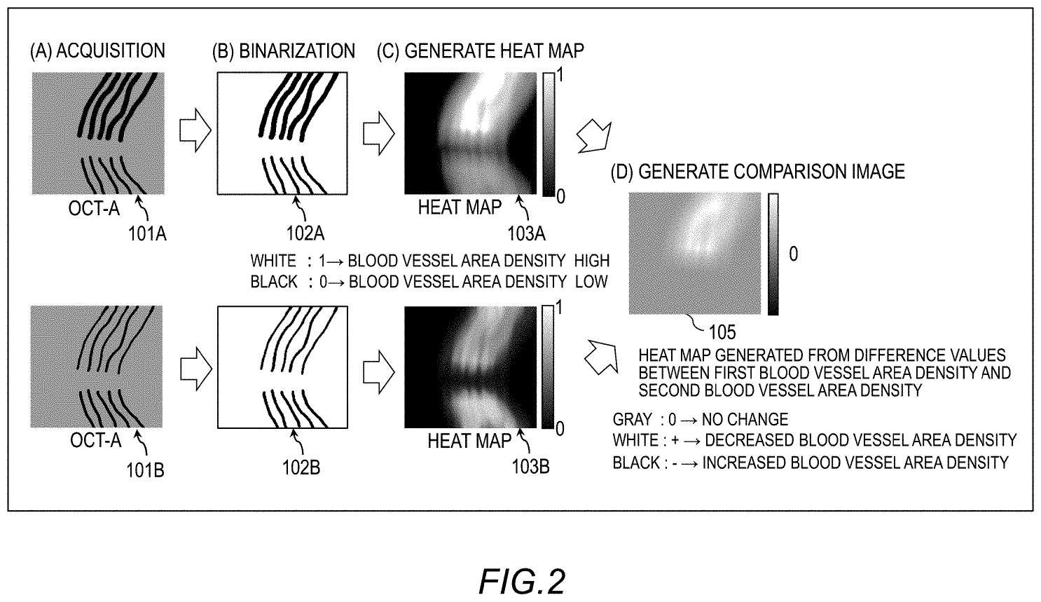

FIG. 2 is a descriptive drawing showing a generation example 2 of heat map data 103 of the eye fundus before and after treatment through PDT performed on a subject eye of a patient suffering from age-related macular degeneration. Description of (A) acquisition to (C) generation of heat map data is omitted due to similarity to FIG. 1. (D) The image processing apparatus generates the comparison image data 105 in which the difference values between the first blood vessel area density and the second blood vessel area density used for generating the heat map data 103A and the heat map data 103B of (C) are visualized.

(D) The comparison image data 105 is difference image data that visualizes the difference values between the first blood vessel area density and the second blood vessel area density in a heat map format. In the heat map data, the difference is taken between the values of the first blood vessel area density within the relevant region of the first angiographic image data 102A and the values of the second blood vessel area density within the same relevant region of the second angiographic image data 102A, and the relevant region is depicted with colors indicating the difference values.

In FIG. 2, the comparison image data 105 that is the difference image data of the blood vessel area density is grayscale image data, for example, where gray represents a pixel value of 0, and the image becomes whiter the more the pixel value increases above 0, indicating that the blood vessel area density has decreased after treatment as compared to before treatment. Also, the more the pixel value decreases below 0, the blacker the image is, indicating that the blood vessel area density after treatment has increased compared to before treatment. The heat map data 105 may be image data in which regions are filled with colors corresponding to the grayscale. However, the configuration is not limited to the comparison image data 105 in which differences in the blood vessel area density are represented by different colors, and image data that represents the difference values through contour lines or image data that represents the difference values as numerical values may be used.

As a result, the comparison image data 105 is displayed by the image processing apparatus or the output destination of the comparison image data 105. Thus, when the comparison image data 105 is displayed, users such as physicians can observe such therapeutic effects. Also, in the comparison image data 105, regions where there is a difference in blood vessel area density before and after treatment are distinguished from regions with no such difference in blood vessel area density, and thus, the user can easily observe differences in blood vessel area density.

In the comparison image data 105, regions where the blood vessel area density has decreased after treatment compared to before treatment are depicted with white, and thus, the user can easily observe the decrease in blood vessel area density (decrease in new blood vessels, reduction in choroid blood vessel diameter, restoration of dilated blood vessels to a normal diameter) as a result of photodynamic therapy (PDT).

<System Configuration Example>

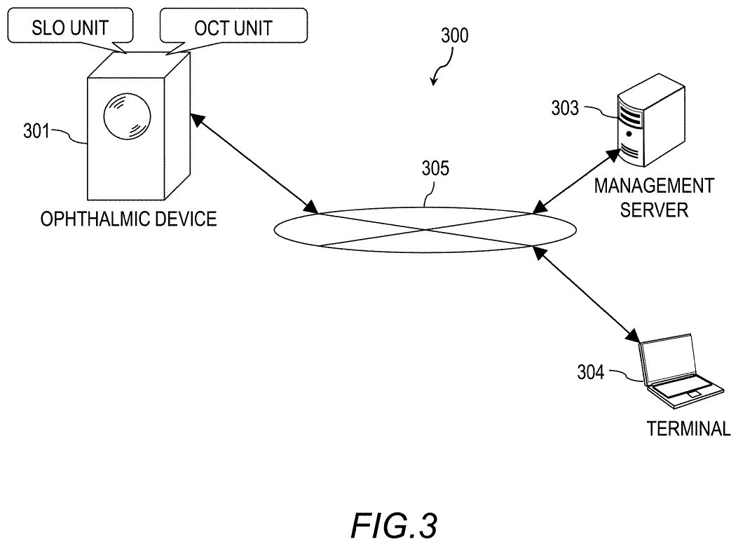

FIG. 3 is a descriptive view showing a configuration example of an ophthalmic system. In the ophthalmic system 300, an ophthalmic device 301, a management server 303, and a terminal 304 are connected in a manner enabling communication therebetween via a network 305 such as a LAN (local area network), a WAN (wide area network), or the internet.

The ophthalmic apparatus 301 has an SLO (scanning laser ophthalmoscope) unit and an OCT unit. The SLO unit scans a laser beam on the subject eye and generates SLO fundus image data of the subject eye on the basis of reflected light from the fundus. The OCT unit generates OCT image data of the fundus through optical coherence tomography. In the present embodiment, the angiographic image data 101 is generated on the basis of the OCT image data.

The management server 303 acquires and stores image data from the ophthalmic apparatus 301, and transmits image data based on a request or image data subjected to image processing to the ophthalmic apparatus 301 and the terminal 304. The terminal 304 receives and displays image data from the management server 303 and transmits, to the management server 303, image data processed by the terminal 304, inputted text information, or the like.

At least one of the ophthalmic apparatus 301, the management server 303, and the terminal 304 can execute the image processing ((A) acquisition to (D) comparison image generation) described with reference to FIGS. 1 and 2. Also, a configuration may be adopted in which at least two computers among the ophthalmic apparatus 301, the management server 303, and the terminal 304 can execute the image processing ((A) acquisition to (D) comparison image generation).

<Computer Hardware Configuration Example>

Next, a computer hardware configuration example will be described. A computer is a collective term for the ophthalmic apparatus 301, the management server 303, and the terminal 304 shown in FIG. 3. If the computer is the ophthalmic apparatus 301, then the SLO unit and the OCT-A unit (not shown) are included.

<Hardware Configuration Example of Computer>

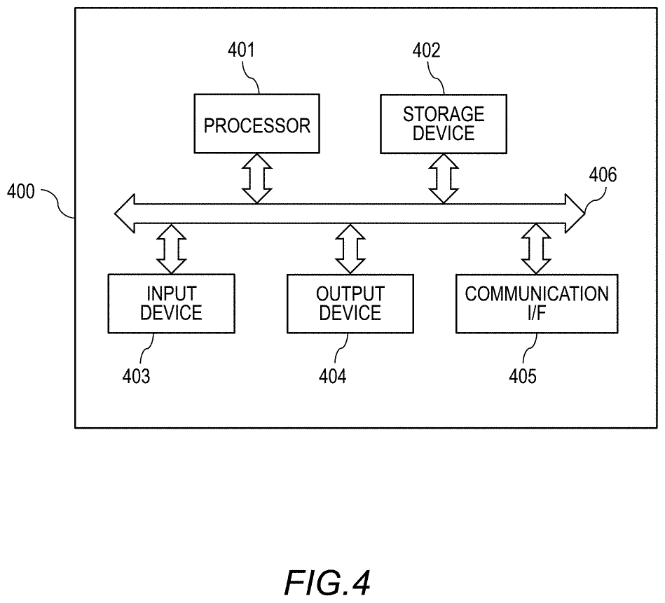

FIG. 4 is a block diagram for illustrating a hardware configuration example of a computer. A computer 400 includes a processor 401, a storage device 402, an input device 403, an output device 404, and a communication interface (communication IF) 405. The processor 401, the storage device 402, the input device 403, the output device 404, and the communication IF 405 are coupled to one another through a bus 406. The processor 401 is configured to control the computer 400. The storage device 402 serves as a work area for the processor 401. The storage device 402 is also a non-transitory or transitory recording medium configured to store various programs and various kinds of data. Examples of the storage device 402 include a read only memory (ROM), a random access memory (RAM), a hard disk drive (HDD), and a flash memory. The input device 403 is configured to input data. Examples of the input device 403 include a keyboard, a mouse, a touch panel, a numeric keypad, a scanner and a sensor. The output device 404 is configured to output data. Examples of the output device 404 include a display, a printer, and a speaker. The communication IF 405 is coupled to the network 305, and is configured to transmit and receive data.

<Functional Configuration Example of Image Processing Apparatus>

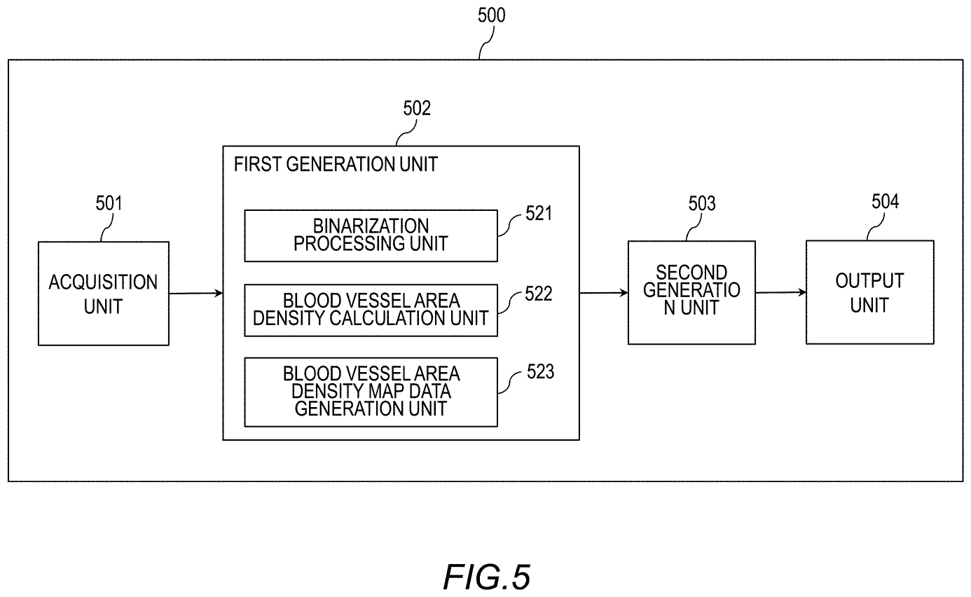

Next, a functional configuration example of the image processing apparatus will be described with reference to FIG. 5. The image processing apparatus is one or more computers 400 that execute at least one of the (A) acquisition to (D) comparison image generation processes described with reference to FIG. 1 or 2. Thus, the image processing apparatus may be realized as an image processing system in which a plurality of computers 400 are linked.

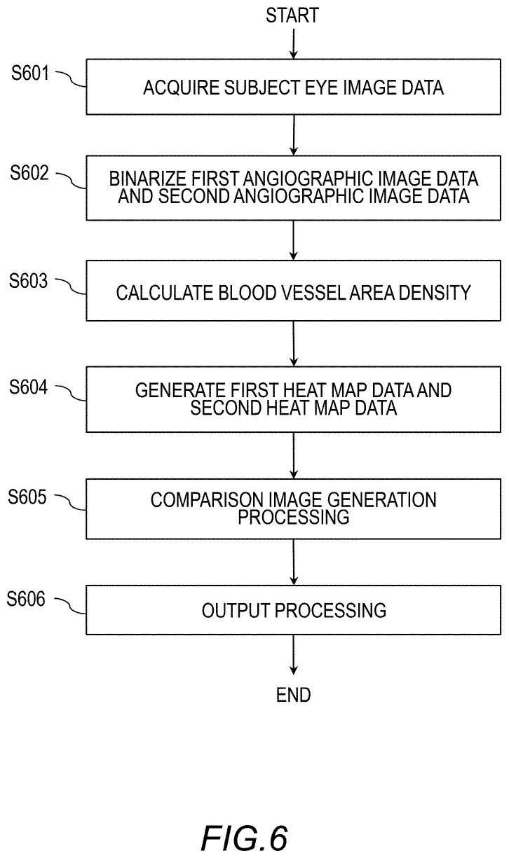

FIG. 5 is a block diagram showing a functional configuration example of the image processing apparatus 500. FIG. 6 is a flowchart showing an example of image processing steps executed by the image processing apparatus 500.

The image processing apparatus 500 has an acquisition unit 501, a first generation unit 502, a second generation unit 503, and an output unit 504. The first generation unit 502 has a binarization processing unit 521, a blood vessel area density calculation unit 522, and a blood vessel area density map data generation unit 523. The acquisition unit 501, the first generation unit 502, the second generation unit 503, and the output unit 504 are specifically realized by a processor 401 executing programs stored in a storage device 402 shown in FIG. 4, for example.

The acquisition unit 501 acquires subject eye image data such as the angiographic image data 101 and SLO fundus image data of a given patient as described with reference to (A) of FIGS. 1 and 2 (step S601). The acquisition unit 501 receives the subject eye image data via the network 305 from another computer 400 having the subject eye image data. Also, if subject eye image data is already stored in the storage device 402 of the image processing apparatus 500, then the acquisition unit 501 reads the subject eye image data from the storage device 402.

The binarization processing unit 521 of the first generation unit 502 subjects the angiographic image data 101 to binarization processing as described in (B) of FIGS. 1 and 2 and outputs the binarized angiographic image data 102 (step S602). Specifically, the binarization processing unit 521 subjects the angiographic image data 101 to binarization processing through discriminant analysis. The binarization processing unit 521 subjects the angiographic image data 101 to binarization processing at a luminance threshold t at which the following formula (1) reaches the maximum value.

w1×w2(m1−m2)² (1)

w1 is the number of pixels with a lower luminance value than the threshold t, where the threshold t is a threshold at which binarization processing is performed. m1 is the average number w1 of pixels. w2 is the number of pixels with a luminance value greater than or equal to the threshold t, where the threshold t is a threshold at which binarization processing is performed. m2 is the average number w2 of pixels. The binarization processing unit 521 is not limited to discriminant analysis and may execute binarization processing at a preset threshold t. The binarization processing unit 521 may execute pre-processing such as luminance adjustment and denoising prior to binarization processing.

The blood vessel area density calculation unit 522 of the first generation unit 502 calculates the blood vessel area density according to the binarized angiographic image data 102 (step S603). Specifically, the blood vessel area density calculation unit 522 performs raster scanning of an averaging filter on a region of a prescribed size (e.g., 100×100 pixel described above) in the angiographic image data 102 subjected to binarization processing, thereby executing a convolution operation through sum-of-product operation of the weighting in the averaging filter and the luminance values of the pixels, for example. The results of the convolution operation are an array of grayscale pixels having a value of 0-1. Each pixel of the convolution operation result indicates the blood vessel area density.

As described in (C) of FIGS. 1 and 2, the blood vessel area density map data generation unit 523 of the first generation unit 502 generates the heat map data 103, for example, as blood vessel area density map data on the basis of the array that is the convolution operation result calculated by the blood vessel area density calculation unit 522 (step S604). Specifically, the blood vessel area density map data generation unit 523 converts each pixel indicating the blood vessel area density that is the convolution operation result from grayscale to RGB colors, for example. The conversion method may be a method in which the pixel is converted to an RGB color value corresponding to the grayscale value with reference to a lookup table, or may be a method in which the RGB color value corresponding to the grayscale value is calculated on the basis of a conversion formula.

As described with reference to (D) of FIGS. 1 and 2, the second generation unit 503 generates comparison image data 104 and 105 (step S605). Specifically, the second generation unit 503 generates the comparison image data 104 or the comparison image data 105 on the basis of the user selecting to display the comparison image data 104 in which two pieces of blood vessel area density map data are arranged side by side, or to display the comparison image data 105 in which the difference values between the two pieces of blood vessel area density data are visualized, for example. Also, the second generation unit 503 may generate both pieces of comparison image data 104 and 105 and switch which to display according to user selection. It is also naturally possible to display both pieces of comparison image data 104 and 105.

The output unit 504 outputs the comparison image data 104 and 105 generated by the second generation unit 503 (step S605). Specifically, the output unit 504 transmits the comparison image data 104 and the comparison image data 105 to the display device of the image processing apparatus 500, or transmits the comparison image data 104 and the comparison image data 105 from the image processing apparatus 500 to another computer 400, for example.

<Display Screen Example>

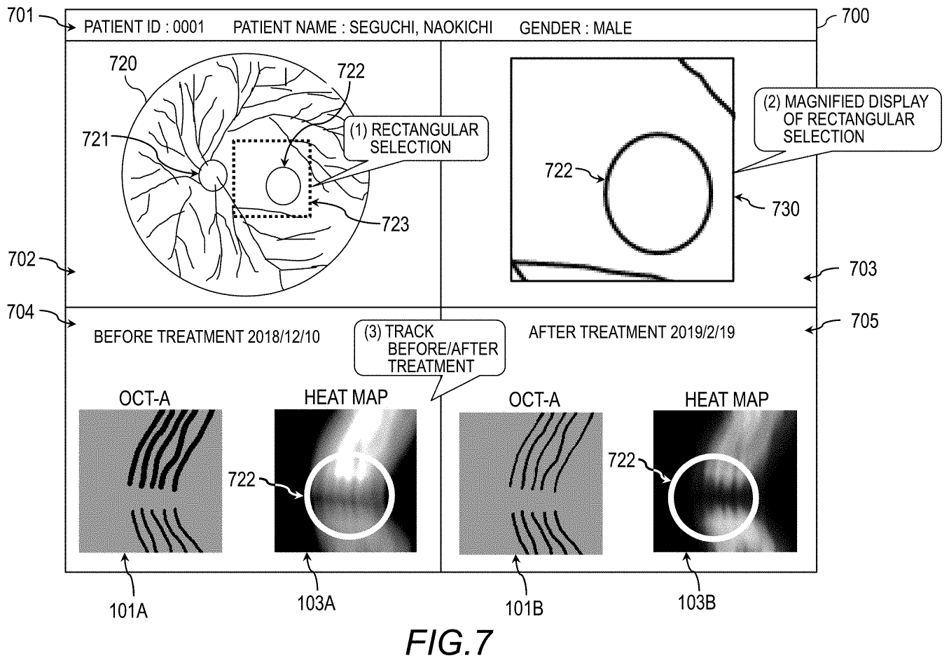

FIG. 7 is a descriptive drawing showing a display screen example. A display screen 700 is displayed in a display (e.g., the display of the management server 303) connected to the output unit 504 or the computer 400 (e.g., the display of the terminal 304) that is the output destination of the output unit 504. The display screen 700 has a patient information display region 701, an SLO fundus image data display region 702, an SLO fundus image data magnified display region 703, a first angiographic image data display region 704, and a second angiographic image data display region 705.

The patient information display region 701 is a region displaying patient information. The patient information is identification information such as the patient ID, patient name, and gender that uniquely identifies the patient.

The SLO fundus image data display region 702 is a region that displays SLO fundus image data 720 (the SLO fundus image data 720 is in this example the SLO fundus image data captured on Feb. 19, 2019 after treatment) captured by the SLO unit of the ophthalmic apparatus 310. The SLO fundus image data 720 is image data attained by capturing a region of the fundus of the subject eye including the optic disc 721, the macula 722, and blood vessels (depicted as line segments).

The SLO fundus image data display region 702 is a region where a rectangular region 723 can be selected. The rectangular region 723 is a rectangular region selected through operation of an input device 403 of the computer 400 where the display screen 700 is displayed. The SLO fundus image data display region 702 also indicates left/right eye identification information 724 indicating whether the subject eye is the right eye or the left eye (left eye in the case of FIG. 7).

The SLO fundus image data magnified display region 703 is a region where the SLO fundus image data 720 is displayed in a magnified view. Specifically, in the SLO fundus image data magnified display region 703, the SLO fundus image data 730 within the rectangular region 723 is displayed in a magnified view.

The first angiographic image data display region 704 is a region that displays the first angiographic image data 101A and the heat map data 103A generated using OCT fundus image data (not shown) captured by the OCT unit of the ophthalmic apparatus 310 on Dec. 10, 2018 prior to treatment. The first angiographic image data 101A is partial angiographic image data of a region of the OCT fundus image data corresponding to the rectangular region 723 designated in the SLO fundus image data 720 among the first angiographic image data of the entire OCT fundus image data. Similarly, the heat map data 103A is partial heat map data of a region of the OCT fundus image data corresponding to the rectangular region 723 among the heat map data of the entire OCT fundus image data.

The second angiographic image data display region 705 is a region that displays the second angiographic image data 101B and the heat map data 103B generated from OCT fundus image data (not shown) captured by the OCT unit of the ophthalmic apparatus 310 on Feb. 9, 2019 after treatment. The second angiographic image data 101B is partial angiographic image data of a region corresponding to the same position as the rectangular region 723 in the second angiographic image data of the entire SLO fundus image data. Similarly, the heat map data 103B is partial heat map data of a region corresponding to the same position as the rectangular region 723 in the heat map data of the entire OCT fundus image data.

Thus, the computer 400 selects the rectangular region 723 from the SLO fundus image data 720, thereby acquiring the partial angiographic image data 101 and the partial heat map data 103 of the region corresponding to the rectangular region 723 from the angiographic image data of the entire subject eye, and displays the first angiographic image data display region 704 and the second angiographic image data display region 705.

As a result, the computer 400 can display the partial angiographic image data 101 and the partial heat map data 103 in conjunction with the selection of the rectangular region 723 in the SLO fundus image data 720. Therefore, it is possible to mitigate misdiagnoses occurring as the result of a discrepancy in the region on which the user wishes to focus among the SLO fundus image data 720, the angiographic image data 101, and the heat map data 103. Also, there is no need to select the region on which the user wishes to focus from the angiographic image data of the entire subject eye, enabling an improvement in convenience to the user.

Additionally, a configuration may be adopted in which difference image data between the heat map data 103A and the heat map data 103B is displayed in the display screen 700. The location subjected to PDT treatment (location irradiated by a laser beam in PDT treatment) may be displayed so as to be superimposed on the difference image data. Also, the location subjected to PDT treatment (location irradiated by a laser beam in PDT treatment) may be displayed so as to be superimposed on the SLO fundus image data 720 and the heat map data 103A and 103B.

Additionally, the computer 400 may display mark data indicating the location of specific tissue within the rectangular region 723 (in the case of FIG. 7, the circular mark data indicating the location of the macula 722) so as to be superimposed on the heat map data 103A and the heat map data 103B. As a result, the user can intuitively tell the corresponding location of the heat map data 103A and 103B in the SLO fundus image data 720.

Also, the image processing apparatus 500 may display together the positions of the heat map data 103A and 103B and the position of the SLO fundus image data 720 so as to be superimposed on each other. The mixture ratio of the superimposition may be modifiable as appropriate through user operation. The image processing apparatus 500 may display together the comparison image data 105, which is the difference image data of the heat map data 103A and 103B, and the position of the SLO fundus image data 720 so as to be superimposed on each other. The mixture ratio of the superimposition may be modifiable as appropriate through user operation.

Also, the image processing apparatus 500 of the embodiment creates heat map data using angiographic image data through OCT angiography, but may create angiographic image data through fluorescence imaging. Also, the image processing apparatus 500 may create heat map data using choroid blood vessel image data attained through image processing of the SLO fundus image data. The choroid blood vessel image data is attained by performing image processing of green SLO fundus image data captured under a green laser beam and red SLO fundus image data captured under red light.

Specifically, the image processing apparatus 500 extracts retinal blood vessels by subjecting the green SLO fundus image data to black hat filter processing. Next, the image processing apparatus 500 removes retinal blood vessels in the red SLO fundus image data from the red SLO fundus image data by filling pixels at the positions of the retinal blood vessels extracted from the green SLO fundus image data through inpainting processing. Through this processing, it is possible to attain the choroid blood vessel image data.

In this manner, according to the above-mentioned image processing apparatus, it is possible to visualize with ease the effects of treatment of an ophthalmic disorder treated through photodynamic therapy using angiographic image data that is an en face image at the depth of the choroid position according to 3-dimensional OCT angiography data. As a result, reliability of follow-up observation of a lesion is improved, and it is possible to mitigate the overlooking or misdiagnosis of the lesion.

In the embodiment above, angiographic image data that is an en face image was used, but the image processing apparatus may create 3-dimensional heat map data through 3-dimensional OCT angiography data of a space including the choroid. In this manner it is possible to have a spatial understanding of the region of the choroid where an ophthalmic disorder is present through 3-dimensionalization.

The present invention is not limited to the content above, and the content above may be freely combined. Also, other aspects considered to be within the scope of the technical concept of the present invention are included in the scope of the present invention.

### EXPLANATION OF REFERENCES

101 Angiographic image data, 102 Binarized angiographic image data, 103 Heat map data, 104,105 Comparison image data, 300 An ophthalmic system, 301 An ophthalmic apparatus, 303 A management server, 304 A terminal, 400 A computer, 401 A processor, 500 A image processing apparatus, 501 An acquisition unit, 502 A first generation unit, 503 second generation unit, 504 An output unit, 521 An binarization processing unit, 522 A blood vessel area density calculation unit, 523 A blood vessel area density map data generation unit

1. An image processing apparatus, comprising:
an acquisition unit that acquires first angiographic image data of a subject eye, and second angiographic image data of the subject eye generated after the first angiographic image data;
a first generation unit that calculates a first blood vessel area density from the first angiographic image data to generate first blood vessel area density map data based on the first blood vessel area density, and that calculates a second blood vessel area density from the second angiographic image data to generate second blood vessel area density map data based on the second blood vessel area density;
a second generation unit that generates comparison image data for comparing the first blood vessel area density map data to the second blood vessel area density map data; and
an output unit that outputs the comparison image data.

2. The image processing apparatus according to claim 1,
wherein the comparison image data includes the first blood vessel area density map data and the second blood vessel area density map data.

3. The image processing apparatus according to claim 1,
wherein the first blood vessel area density map data is first heat map data represented in heat map format, and
wherein the second blood vessel area density map data is second heat map data represented in heat map format.

4. The image processing apparatus according to claim 1,
wherein the comparison image data is difference image data based on a difference value between the first blood vessel area density and the second blood vessel area density.

5. The image processing apparatus according to claim 4,
wherein the difference image data is difference heat map data created on the basis of the difference value.

6. The image processing apparatus according to claim 1,
wherein the first angiographic image data and the second angiographic image data are angiographic image data attained by OCT angiography.

7. The image processing apparatus according to claim 1,
wherein the first angiographic image data and the second angiographic image data are choroid blood vessel image data.

8. The image processing apparatus according to claim 6,
wherein the first angiographic image data is generated on the basis of first OCT image data captured of the subject eye prior to treatment, and the second angiographic image data is generated on the basis of second OCT image data captured of the subject eye after treatment.

9. The image processing apparatus according to claim 1,
wherein the second generation unit superimposes mark data indicating a position of specific tissue of the subject eye on the first blood vessel area density map data and the second blood vessel area density map data, or the comparison image data.

10. The image processing apparatus according to claim 1,
wherein the second generation unit generates an image attained by superimposing the comparison image data on fundus image data.

11. An image processing method,
wherein a processor executes:
acquisition processing for acquiring first angiographic image data of a subject eye, and second angiographic image data of the subject eye generated after the first angiographic image data;
first generation processing for calculating a first blood vessel area density from the first angiographic image data to generate first blood vessel area density map data based on the first blood vessel area density, and for calculating a second blood vessel area density from the second angiographic image data to generate second blood vessel area density map data based on the second blood vessel area density;
second generation processing for generating comparison image data for comparing the first blood vessel area density map data to the second blood vessel area density map data; and
output processing for outputting the comparison image data.

12. A non-transitory recording medium storing thereon an image processing program for causing a processor to execute the image processing method according to claim 11.
